(19) United States
(12) Patent Application Publication
(10) Pub. No.: US 2014/0276138 A1
(43) Pub. Date: Sep. 18, 2014

(54) SENSING GUIDEWIRES WITH A CENTERING ELEMENT AND METHODS OF USE THEREOF

(71) Applicant: VOLCANO CORPORATION, San Diego, CA (US)

(72) Inventor: Bret Millett, Folsom, CA (US)

(73) Assignee: VOLCANO CORPORATION, San Diego, CA (US)

(21) Appl. No.: 14/208,286

(22) Filed: Mar. 13, 2014

Related U.S. Application Data
(60) Provisional application No. 61/778,895, filed on Mar. 13, 2013.

(51) Int. Cl. A61B 5/00; A61B 5/026; A61B 5/0215

(52) U.S. Cl. CPC A61B 5/6851; A61B 5/0215; A61B 5/026
USPC 600/486; 600/585; 600/505

(57) ABSTRACT

The invention generally relates to sensing guidewires with a centering element and methods of use thereof. In certain aspects, guidewires of the invention include a core member sized to fit within a vessel. A sensor is coupled to the core member. A centering element including a retracted and deployed configuration that is coupled to the core member such that upon deployment of the centering element, the sensor on the core member is located within a center of a lumen.

### RELATED APPLICATION

The present application claims the benefit of and priority to U.S. provisional patent application Ser. No. 61/778,895, filed Mar. 13, 2013, the content of which is incorporated by reference herein in its entirety.

### FIELD OF THE INVENTION

The invention generally relates to sensing guidewires with a centering element and methods of use thereof.

### BACKGROUND

Cardiovascular disease frequently arises from the accumulation of atheroma material on inner walls of vascular lumens, particularly arterial lumens of the coronary and other vasculature, resulting in a condition known as atherosclerosis. Atherosclerosis occurs naturally as a result of aging, but it may also be aggravated by factors such as diet, hypertension, heredity, and vascular injury. Atheroma and other vascular deposits restrict blood flow and can cause ischemia that, in acute cases, can result in myocardial infarction. Atheroma deposits can have widely varying properties, with some deposits being relatively soft and others being fibrous and/or calcified. In the latter case, the deposits are frequently referred to as plaque. Depending on the level of atheroma deposits which occlude the vessel, the diseased vessel is often called partially-occluded or total occluded vessel.

It is often desirable to take pressure and flow measurements within the vessel occluded by the atheroma. Previous techniques required introducing a catheter with pressure and flow sensors into the vessel. However, catheters are often too large in diameter to reach the vessel of interest or their size interferes with blood flow resulting in inaccurate pressure and flow measurements. In order to improve and streamline procedures, guidewires, which have significantly smaller diameters than catheters, have been designed to include miniature pressure and flow sensors near the distal tip of the guidewires. The guidewires generally include a core wire. Conductive wires are then run along the length of the core. A hypotube then covers the wires, and terminal ends of the wires are coupled to a sensor, e.g., a pressure or flow sensor. These guidewire are less disruptive to blood flow and are able to provide more accurate pressure and flow reading.

A problem with existing pressure and flow guidewires is that there position within the vessel is arbitrary. They may be positioned in the center or they may be positioned along the side. Flow within a vessel is different at the center and at the edges. Therefore, in order to obtain optimum and consistent vessel-to-vessel measurements, readings should be taken at the center of a vessel. If positioned along a side of a vessel, less than optimal readings are obtained. Additionally, there is no vessel-to-vessel consistency among readings because the readings are taken at different locations within the vessel.

### SUMMARY

The invention provides sensing guidewires with a centering element. In this manner the sensor on the guidewire is always making readings from a center of a vessel, ensuring optimal readings are obtained and ensuring vessel-to-vessel consistency among readings. Aspects of the invention are accomplished using a centering element coupled to the guidewire. The centering element is deployed once the guidewire is positioned at a target location. The deployed centering element centers the sensor prior to imaging.

Guidewires of the invention include a core member sized to fit within a vessel. A sensor is coupled to the core member. A centering element including a retracted and deployed configuration that is coupled to the core member such that upon deployment of the centering element, the sensor on the core member is located within a center of a lumen. Any design or configuration of a centering member may be used with methods of the invention. In certain embodiments, the centering element surrounds the guidewire such that it deploys a same distance on all sides of the guidewire. In certain embodiments, the centering element includes a plurality movable struts.

The sensor(s) and centering elements may be placed anywhere along the guidewire. Typically, the sensor and the centering element are positioned along a distal portion of the guidewire. The centering element can be co-located with the sensor or located proximal or distal to the sensor. the sensor is positioned on a distal portion of the guidewire.

Any type of sensor can be connected to guidewires of the invention and the type of measurement will determine the type of sensor used. In certain embodiments, only a single sensor is connected to the guidewire. In other embodiments, multiple sensors are connected to the guidewire. All of the sensors may be the same. Alternatively, the sensors may differ from each other and measure different characteristics inside a vessel. Exemplary sensors are pressure, flow, and temperature sensors. Any type of pressure sensor may be used with guidewires of the invention. In certain embodiments, the pressure sensor includes a crystalline semi-conductor material. Any type of flow sensor may be used with guidewires of the invention. In certain embodiments, the flow sensor includes an ultrasound transducer.

Another aspect of the invention provides methods for measuring a characteristic inside a vessel. The methods involve providing a sensing guidewire. The guidewire includes a core member sized to fit within a vessel. A sensor is coupled to the core member. A centering element including a retracted and deployed configuration that is coupled to the core member such that upon deployment of the centering element, the sensor on the core member is located within a center of a lumen. Methods of the invention additionally involve inserting the guidewire into a vessel, centering the sensor within the vessel via deployment of the centering element, and using the sensor to measure a characteristic inside the vessel.

### BRIEF DESCRIPTION OF THE DRAWINGS

FIG. 1 depicts a side view of an adjustable sensing guidewire of the invention according to one embodiment.

FIG. 2 is a schematic illustration showing use of an adjustable guidewire of the invention according to one embodiment during a catheterization procedure on a patient.

FIGS. 3A and 3B depict a distal portion of the guidewire and show the distal portion in the relaxed state and the compressed state.

FIGS. 4A and 4B exemplify the compression of the coil segment, which includes a compressible bendable portion.

FIG. 5 exemplifies the various tip adjustments one can accomplish with the guidewire of the invention according to certain embodiments.

FIGS. 6 and 7 illustrate an exemplary sensor configuration and sensor housing according to certain embodiments.

FIG. 8 illustrates a configuration of electrical connector wires around the core member according to certain embodiments.

FIG. 9 illustrates the electrical connector wires embedded with the core member according to certain embodiments.

FIG. 10 illustrates an ideal connector for the electrical connector wires according to certain embodiments.

FIG. 11 depicts a non-sensing guidewire according to certain embodiments.

FIG. 12 is a diagraph showing a sensing guidewire of the invention with a centering element in a retracted configuration.

FIG. 13 is a diagraph showing a sensing guidewire of the invention with a centering element in a deployed configuration.

FIG. 14 is a diagraph showing a sensing guidewire of the invention with a centering element in a retracted configuration within a vessel. The guidewire is located close to one of the vessel walls and not in the center of the vessel. FIG. 15 is a diagraph showing a sensing guidewire of the invention with a centering element in a deployed configuration within a vessel. The guidewire is centered within the vessel.

FIG. 16 shows a representation of a strut for an umbrella centering element.

FIG. 17 shows a representation of a mushroom centering element.

FIG. 18 is a system diagram according to certain embodiments.

### DETAILED DESCRIPTION

The invention generally relates to sensing guidewires with a centering element and methods of use thereof. In certain aspects, guidewires of the invention include a core member sized to fit within a vessel. A sensor is coupled to the core member. A centering element including a retracted and deployed configuration that is coupled to the core member such that upon deployment of the centering element, the sensor on the core member is located within a center of a lumen.

FIG. 12 shows a sensing guidewire 1000 of the invention. The guidewire 1000 includes a core member 1010, a sensor 1020 and a centering element 1030. FIG. 12 shows the centering element 1030 is a retracted configuration, also a delivery configuration. FIG. 13 shows the centering element in a deployed configuration. FIGS. 14-15 show how the centering element works. FIG. 14 shows that prior to deployment within a vessel 1040, the guidewire 1000 is not centered and its position in the vessel is arbitrary. FIG. 15 shows that upon deployment of the centering element 1030, the guidewire 1000 becomes centered in the vessel 1040.

Sensing guidewires are generally known in the art, and are described for example in U.S. Pat. Nos. 6,106,476; 6,106,476; 5,125,137; 6,551,250; and 5,873,835, the content of each of which is incorporated by reference herein in its entirety.

FIG. 1 shows, in more detail, a guidewire 5 of the invention according to certain embodiments. The guidewire 5 includes a flexible elongate member 100 having a proximal portion 102 and a distal portion 104. The guidewire 5 may have an average diameter of about 0.035″ and less. The flexible elongate member 100 typically includes an elongate shaft 116 and a coil segment 112. The flexible elongate shaft 116 can be formed of any suitable material such as stainless steel, nickel and titanium alloy (Nitinol, polyimide, polyetheretherketone or other metallic or polymeric materials and having a suitable wall thickness, such as, e.g., 0.001″ to 0.002″. This flexible elongate shaft is conventionally called a hypotube. In one embodiment, the hypotube may have a length of 130 to 170 cm. The guidewire further includes a core member 150 disposed within a lumen of the elongate member 100. The core member 150 extends from the proximal portion to the distal portion of the flexible elongate member 100 to provide the desired torsional properties to facilitate steering of the guidewire in the vessel and to provide strength to the guidewire and prevent kinking. The core member can be formed of a suitable material such as stainless steel, nickel and titanium alloy (Nitinol), polyimide, polyetheretherketone, or other metallic or polymeric materials.

The elongate body member 100 further includes an elongate shaft 116 operably coupled to the coil segment 112. The elongate shaft 116 defines a lumen extending from the proximal portion 102 to the distal portion 104. The coil segment 112 also defines a lumen extending therethrough. The core member or wire 150 extends through the lumens of the elongate shaft 116 and coil segment 112 and couples or is affixed to the distal portion 104 at a point on the distal portion 104 (see FIGS. 5 and 7). Preferably, the core member 150 couples to a point on the distal portion that is distal to the coil segment 112. In certain embodiments, the core member 150 couples to an inner surface of the distal tip 110 as shown in FIG. 5. Alternatively, the core member 150 can couple to an inner surface of a housing 120 located on the distal portion 104.

The distal portion 104 of flexible elongate member 100 may include one or more coil segments 112. The coil segments 112 can vary in length along the elongate member 100. The coil segment 112 provides additional flexibility to the elongate member 100. Suitable materials for the coil segment 112 include stainless steels, radiopaque metals, platinum alloys, palladium alloys, and any other metals or alloys. The longer the length of the coil segment 112 is along the flexible elongate member 100 the greater the flexibility. In certain embodiments, the coil segment 112 is divided into to two regions, the tip coil and the proximal coil. The tip coil is a portion of the coil segment 112 closer to a distal tip 110 of the elongate member 100. The proximal coil 112 is a portion of the coil segment closer to the elongate shaft 116. The spacing between coils of the coil segment 112 can increase or decrease the flexibility of the coil segment 112. For example, a tightly wound coil, i.e. minimum spacing between coils, increases rigidity of the coil segment and a loosely wound coil, i.e. increased spacing between coils, increase flexibility of the coil segment.

According to certain embodiments, at least a portion of the coil segment 112 includes a compressible bendable portion that is configured to bend or move relative to the longitudinal axis x of the elongate member 100. The compressible portion of coil segment 112 includes increased coil spacing. In certain embodiments, the coil spacing of the compressible portion is sufficient to bend the coils from straight to approximately perpendicular to the longitudinal axis when the coils are compressed. In certain embodiments, the coil spacing of the compressible portion is greater than about 15% spacing (which is the spacing to coil ratio in the compressible region).

The coil segment 112 defines a lumen and the bendable portion of the coil segment 112 is compressible from a relaxed state, as shown in e.g. FIGS. 1 and 3A, to a compressed state, as shown in e.g. FIG. 3B. As the coil segment 112 is being compressed, the coil segment 112 bends away from a longitudinal axis x of the elongate body 100 thus causing portions of the elongate body 100 distal to the coil segment to move in the direction of the coil segment 112. Thus, the bendable portion of the coil segment 112 causes localized bending of the elongate body 100 at the bendable portion of the coil segment 112. The coil segment 112 and expansion and compression of the coil segment 112 are described in more detail in reference to FIGS. 3A-5 hereinafter.

In certain embodiments, the distal portion 104 of the flexible elongate member 100 includes a distal tip 110. The tip 110 may be rounded into a dome-like shape. This allows the guidewire to follow the curve of the vessel and is generally called an atraumatic tip. In certain embodiments, a sensor, such as an ultrasound transducer for measuring flow, is coupled to the distal tip 110. The core member 150 extending through a lumen of the flexible elongate member 100 may connect to an inner surface of the distal tip. In certain embodiments and as shown, a sensor housing 120 couples to and/or forms the distal tip 110 of the flexible elongate member 1000. Alternatively, the coil segment 112 can be coupled to the distal tip 110 of the flexible elongate member 100, as shown in FIG. 11. FIG. 11 depicts a non-sensing guidewire according to certain embodiments.

A sensor housing 120 may be positioned on the elongate member 100. The sensor housing 120 includes a housing body that defines a lumen. One or more cavities may be shaped into the walls of the sensor housing to form windows for sensors disposed or mounted therein. The sensor housing is preferably positioned between the coil segment 112 and the distal tip 110. In certain embodiments, the sensor housing 120 directly couples to the distal tip 110. In other embodiments, another coil segment may be between the sensor housing 120 and the distal tip 110. This additional coil segment provides for a softer, more flexible distal end. In this manner, the sensor housing 120 is sandwiched between coil segments. With this positioning, the sensor housing 120 moves along with the compressible bending portion of the coil segment when it is compressed from a relaxed state to the compressed state (See FIGS.3-5). Optionally and as shown, the guidewire 21 includes a sensor 114, such as a pressure sensor, disposed within a sensor housing 120 between the coil segment 112 and the distal tip 110. Suitable sensors for use in guidewire of the invention are described hereinafter. The sensor housing 120 can be made of substantially the same material as the elongate shaft, which includes, e.g. stainless steel, nickel and titanium alloy (Nitinol), polyimide, polyetheretherketone or other metallic or polymeric materials. The sensor housing 120 is shown in more detail in FIGS. 6 and 7 and discussed in more detail hereinafter.

The coil segment 112 may be coupled to the flexible elongate shaft 116, sensor housing 120, or distal tip 110 using any suitable design and/or manufacturing techniques. For example, the flexible elongate shaft may be coupled to the coil segment by solder or adhesive. In one embodiment, the ends of the coil segment are integrated into the connected ends of the elongate shaft 116 and sensor housing 120. For example, the elongate shaft and sensor housing may include cut-outs, such as the cut-outs 160 shown in FIG. 6, that mate with a portion of the coil segment 120. An adhesive may be applied to the mated portion of coil segment 160 in the elongate shaft 116 or sensor housing 120 to increase the bond between the components. In an alternative embodiment, a thin-wall tubing, such as a polymide tubing, can be placed behind the joints connecting the sensor housing 120, coil segment, and elongate shaft. Using a thin-wall tubing, allows one to create an adhesive/solder free path for the core member 150.

The proximal portion 102 of the elongate body 100 is the portion of the guidewire left outside a patient during a procedure for handling by the operator. The proximal portion 102 includes a gripping member 118 coupled to the elongate shaft 116 of the elongate body 100. The gripping member 118 allows a user to move the elongate shaft 116 towards the distal tip 110 relative to the core member 150. The gripping member 118 defines a lumen that receives a portion of the core member 150 there through. The gripping member 118 and the elongate shaft 116 are configured to slide in the distal and proximal directions relative to the core member 150, which remains fixed to a point on the distal portion 104 that is distal to the coil segment 112.

A proximal end of the core member 150 may be connected to a handle. The operator can hold the handle and slideably move the gripping member 118 and elongate shaft 116 over and along the core member 150. This embodiment is ideal for adjustable guidewires of the invention that do not include sensors. Alternatively and as shown, the proximal end of core member 150 can be removeably coupled to a connector housing 106. In addition to receiving the proximal end of the core member 150, the connector housing 106 may also removeably connect to and receive one or more electrical connection wires (not shown) that run the length of the elongate body 100 and connect to one or more sensors on the distal portion 102. This removable connection allows one to disconnect the guidewire from the connector housing 106 when placing a catheter over the guidewire and reconnect the guidewire thereafter to prove electrical communication to the sensors. The connector housing 106 may include one or more electrical connections that mate with the electrical conductor wires. In certain embodiments, at least a portion of the core member is operably associated with one or more electrical conductor wires. For example, the proximal end of the core wire 150 can form an electrical male connector 162 (as shown in FIG. 10) with the one or more electrical conductor wires that mate with an electrical female connector within the connector housing 106. The connector housing 106 may be connected to an output connector 73 via a cable 108. The output connector 73 is configured to transmit signals from one or more sensors to an instrument, such as a computing device or EKG monitor (described and shown in FIG. 2).

In certain embodiments, a proximal end of the elongate body 100 can be coupled to a torque element that causes rotation of the elongate body 100. In order to provide uniform rotation of the elongate body 100 (e.g. simultaneous rotation of the core member 150, coil segment 112, elongate shaft 116, ect.), the gripping member 118 may include a locking element that fixes the elongate shaft 116 relative to the core member 150. The locking element prevents unintended rotation of the elongate member 116 relative to the core member 150.

FIG. 2 depicts a guidewire 5 of the present invention having sensing capabilities, such as a pressure sensor, that is adapted to be in conjunction for a catheterization procedure to treat a patient 22 lying on a table or a bed 23. A distal portion of the elongate member 100 is disposed within the patient 22. The elongate member 100 is used with apparatus 24 which consists of a cable 26 which connects the elongate member 100 to an interface box 27. Interface box 27 is connected by another cable 28 to a computing device 29. The computing device may have a video screen 31 that can display ECG measurements obtained from sensors on the elongate member 100. For example, the ECG measurements may appear as traces 32, 33 and 34.

The coil segment 120 of the elongate member 100 includes a compressible, bendable portion 210 as shown in, e.g. FIGS. 3A-4B. The compressible bendable portion 210 is configured to bend away from a longitudinal axis of the elongate member 100 upon distal movement of the elongate shaft 116 (which is actually part of the tip coil as shown) relative to the core member 150. FIGS. 3A and 3B depict a distal portion of the elongate member 100 and show the compressible bendable portion 210 in the relaxed state 212 and the compressed state 214. As shown in FIGS. 3A and 3B, the elongate member 100 includes an elongate shaft 116 and a core member 150 extending from and disposed within the elongate shaft 116. Although not shown in FIGS. 3A and 3B, the core member 150 couples to an inner surface of the distal tip 110. The elongate member 100 further includes a compressible, bendable coil segment 210 having a proximal end coupled to the elongate shaft 116 and distal end coupled to a sensor housing 120. FIG. 3A shows the compressible, bendable coil segment 210 in the relaxed state 212. Movement of the elongate shaft 116 relative to the core member 150 and in the distal direction from point 215A to point 215B compresses the bendable coil segment 210 from the relaxed state 212 to the compressed state 214. Compression of the bendable coil segment 210 causes at least a portion the coil segment 120 to bend relative to a longitudinal axis. As shown in 3A and 3B, the sensor housing 120 coupled to the compressible bendable portion 210 moves away from the longitudinal axis x along with the compressed coil segment.

FIGS. 4A and 4B further illustrate the compression of the coil segment 112 that includes a compressible bendable portion 210. The coil segment 112 consists of a wire or other material wound about a longitudinal axis x to form a coils 220. As shown in FIGS. 4A and 4B, the coil segment 112 includes a compressible bendable portion 210 between two more rigid portions 208. The compressible bendable portion 210 has a wider coil spacing 218 than the coil spacing 222 of rigid portions 218. In addition, the amount of spacing dictates the level of bending. Because the coil spacing 222 of the rigid portion 208 is decreased, the rigid portions 208 are not as flexible as the bendable portion 210. The rigid portions 208 are coupled to the elongate shaft 116 and sensor housing 120. The rigid portions 208 provide a moderate transition from the flexibility of the elongate shaft 116 to the flexibility of the bendable portion 210. In certain embodiments, the coil spacing 218 of the bendable portion 210 is greater than a 15% spacing to coil ratio. FIG. 4A shows the coil segment 112 in the relaxed state 212, in which the coil segment aligns with the longitudinal axis x. FIG. 4B shows the coil segment 112 in the compressed state 214, in which the coil segment bends away from the longitudinal axis.

As further shown in FIGS. 4A to 4B, as the elongate shaft 110 moves from point A to point B (relative to core member 150 not shown), the coil segment 112 compresses from a relaxed state 212 to a compressed state 214. In the compressed state 214, the coils are compressed together, thus causing the coils to bend in a direction in accordance to the coil winding angle. As shown, the bendable portion 210 bends significantly more than the rigid portions 208. Depending on the amount of bending desired, one can change the spacing and or the length of the bendable portion 210. This allows one to create guidewires with adjustable tips with varying bendability ranges.

FIG. 5 exemplifies the various tip adjustments one can accomplish by moving the elongate shaft 116 in the distal and proximal directions relative to core member 150. As shown in FIG. 5, by moving the elongate shaft as indicated by arrow W, one can achieve a range of curvature (i.e. bending) of the distal portion as indicated by arrows Y and Z. This range of motion of the distal portion 104 greatly improves the guidewire's performance in vivo. As shown in FIG. 5, the sensor housing 120 and sensors 114 move in a direction away from the longitudinal axis x of the elongate member 100 along with the coil segment 120. This allows an operator to better position the sensors 114 within the vessel or vasculature to obtain measurements. Accordingly, by adjusting the distal portion 104 to re-position the sensors 114 within the vessel, one can obtain better intraluminal measurements, such as pressure and flow measurements, than without the adjustment. In one embodiment, the distal portion is adjusted to place a pressure sensor within a body lumen into an optimal position for measuring intraluminal fluid pressure. In another embodiment, the distal portion is adjusted to place a flow sensor within a body lumen into an optimal position for measuring intraluminal fluid flow.

FIG. 5 also provides a cross-sectional view of the distal portion 104, which shows the core member 150 disposed within the elongate member 100. The core member 150 includes a proximal portion 150b and a distal portion 150 a. Optionally and as shown, the core member 150 may taper in diameter from the proximal portion 150b to the distal portion 150c. In this manner, the distal portion 150c of the core member 150 is more flexible than the proximal portion 150b of the core member 150 and the distal portion 150c of the core member 150 is able to bend away from the longitudinal axis x along with the elongate member 100. As shown, the core wire 150 is coupled to the distal end 110 of the elongate member 100. Alternatively, the core member 150 could couple to a proximal end of the sensor housing 120 or another point along the sensor housing 120. As further shown in FIG. 5, the core member 150 may be operably associated with one or more electrical conductor wires that couple to sensors 114. The electrical conductor wires 300 transmit and receive signals from the sensors 114. The electrical conductor wires 300 as associated with the core member 150 are further shown in FIGS. 8-9.

In certain aspects, the distal portion 104 of the elongate member 100 includes one or more sensors 114. The sensors 114 provide a means to obtain intraluminal measurements within a body lumen and are connected to one or more electrical conductor wires 300, which transmit and receive signals from the sensors 114. For example, the guidewire of the invention can include a pressure sensor, a flow sensor, a temperature sensor or combinations thereof. Preferably, the guidewire is a combination guidewire that includes both a pressure sensor and a flow sensor. Pressure sensors can be used to measure pressure within the lumen and flow sensors can be used to measure the velocity of blood flow. Temperature sensors can measure the temperature of a lumen. A guidewire with both a pressure sensor and a flow sensor provides a desirable environment in which to calculate fractional flow reserve (FFR) using pressure readings, and coronary flow reserve (CFR) using flow readings.

The ability to measure and compare both the pressure and velocity flow and create an index of hyperemic stenosis resistance significantly improves the diagnostic accuracy of this ischemic testing. It has been shown that distal pressure and velocity measurements, particularly regarding the pressure drop-velocity relationship such as Fractional Flow reserve (FFR), Coronary flow reserve (CFR) and combined P-V curves, reveal information about the stenosis severity. For example, in use, the guidewire may be advanced to a location on the distal side of the stenosis. The pressure and flow velocity may then be measured at a first flow state. Then, the flow rate may be significantly increased, for example by the use of drugs such as adenosine, and the pressure and flow measured in this second, hyperemic, flow state. The pressure and flow relationships at these two flow states are then compared to assess the severity of the stenosis and provide improved guidance for any coronary interventions. The ability to take the pressure and flow measurements at the same location and same time with the combination tip sensor, improves the accuracy of these pressure-velocity loops and therefore improves the accuracy of the diagnostic information.

A pressure sensor allows one to obtain pressure measurements within a body lumen. A particular benefit of pressure sensors is that pressure sensors allow one to measure of fractional flow reserve (FFR) in vessel, which is a comparison of the pressure within a vessel at positions prior to the stenosis and after the stenosis. The level of FFR determines the significance of the stenosis, which allows physicians to more accurately identify hemodynamically relevant stenosis. For example, an FFR measurement above 0.80 indicates normal coronary blood flow and a non-significant stenosis. Another benefit is that a physician can measure the pressure before and after an intraluminal intervention procedure to determine the impact of the procedure.

A pressure sensor can be mounted on the distal portion of a flexible elongate member. In certain embodiments, the pressure sensor is positioned distal to the compressible and bendable coil segment of the elongate member. This allows the pressure sensor to move away from the longitudinal axis and coil segment as bended. The pressure sensor can be formed of a crystal semiconductor material having a recess therein and forming a diaphragm bordered by a rim. A reinforcing member is bonded to the crystal and reinforces the rim of the crystal and has a cavity therein underlying the diaphragm and exposed to the diaphragm. A resistor having opposite ends is carried by the crystal and has a portion thereof overlying a portion of the diaphragm. Electrical conductor wires can be connected to opposite ends of the resistor and extend within the flexible elongate member to the proximal portion of the flexible elongate member. Additional details of suitable pressure sensors that may be used with devices of the invention are described in U.S. Pat. No. 6,106,476; . U.S. Pat. No. 6,106,476 also describes suitable methods for mounting the pressure sensor 104 within a sensor housing.

In certain aspects, the guidewire of the invention includes a flow sensor. The flow sensor can be used to measure blood flow velocity within the vessel, which can be used to assess coronary flow reserve (CFR). The flow sensor can be, for example, an ultrasound transducer, a Doppler flow sensor or any other suitable flow sensor, disposed at or in close proximity to the distal tip of the guidewire. The ultrasound transducer may be any suitable transducer, and may be mounted in the distal end using any conventional method, including the manner described in U.S. Pat. No. 5,125,137, 6,551,250 and 5,873,835.

FIGS. 6 and 7 illustrate an exemplary sensor configuration and sensor housing 120 of the guidewire of the invention. As shown in FIGS. 6 and 7, the distal portion of the elongate member 100 includes a flow sensor 400 and the pressure sensor 402. The flow sensor 400 is located near the distal tip 110 of the elongate member 100. The flow sensor 400 may be an ultrasound array. As shown, the flow sensor 400 has a ferrule shape that allows the core member 150 to extend there through and couple to the distal tip 110 of the elongate member 100. The pressure sensor 402 is mounted in a cavity 500 of the sensor housing 120. The cavity 500 includes an opening 501 that exposes the pressure sensor 402 to external environments so that it can obtain pressure measurements.

In certain embodiments, one or more electrical connection wires are coupled to one or more sensors. The electrical connection wires can include a conductive core made from a conductive material, such as copper, and an insulative coating, such as a polymide, fluoropolymer, or other insulative material. The electrical connection wires extend from one or more sensors located on the distal end of the guidewire, run down the length of the guidewire, and connect to a connector housing at a proximal end.

Any suitable arrangement of the electrical connection wires through the length of the elongate member can be used. The arrangement of electrical connection wires must provide for a stable connection from the proximal end of the guidewire to the distal end of the guidewires.

In addition, the electrical connection wires must be flexible and/or have enough slack to bend and/or move with the adjustable distal portion without disrupting the sensor connection. In one embodiment, the electrical connections run next the core member within the lumen of the elongate member. In another embodiment, the electrical connection wires 300 are wrapped around the core member 150, as shown in FIG. 8.

In yet another embodiment, the electrical connector wires 300 are embedded on the core member 150. For example, the electrical connection wires 300 are wrapped around the core member 150 (as shown in FIG. 8) and then covered with a polymide layer 310 as shown in FIG. 9. At a distal end of the core member 150 near the sensors, the polymide layer 310 can be dissected away, as shown in section 312, which frees the wires to extend and connect to their respective sensors. The length of the electrical connector wire 300 running free from the core member 150 and connected to the sensor should have enough slack/flexibility to remain connected to the sensor during bending of the adjustable tip.

As discussed, sensing guidewires of the invention include a centering element. The centering element can include a stent-like device, a strut-like device, a spring-strut-like device, an umbrella-like device, a mushroom-like device, or other device that allows the guidewire to be centered within a vessel. The centering element inserted in a retracted or delivery configuration and is opened to an expansion or deployed configuration once at a target site within the vessel. Deployment may be by a control wire coupled to the centering element that is controlled by an operator. FIG. 16 shows a representation of a strut for an umbrella centering element 1003. It is shown as an open-end, stent-like device in the deployed configuration. FIG. 17 shows a representation of a mushroom centering element.

As discussed, a proximal end of the electrical connection wires 300 connects to a connector housing, such as connector housing 106 in FIG. 1. In certain embodiments, the electrical connector wires 300 are joined together to form a male connector at a proximal end. The male connector mates with a female connector of the connector housing. FIG. 10 depicts an exemplary male connector for use in devices of the invention. The termination of the male connector is performed by a metal deposition process at a proximal section 162 of the core member 150. An area made up of intermediate areas 150a, 150b, 150c and 150d is masked and metal is deposited at areas 130a, 130b, 130c, 130d and 130e. A process of this nature is described in U.S. Pat. No. 6,210,339, incorporated herein by reference in its entirety. The deposited metal (or any conductive material) permanently adheres or couples to the exposed conductive wires at points 140a-e where the polyimide layers were removed. After the masking material 150a-d is removed, there are five independent conductive stripes 130a-e, each connected to a different respective electric wire. Because of the precision nature of the winding process as well as the masking and metal deposition processes, a male connector is made that is short in length, yet very reliable, in mating with a female connector and cable. Any metallizing process is conceived here, including the metallizing of the entire section 162, followed by the etching of the metal material at 150a, 150b, 150cand 150d . Alternatively, conductive bands may be coupled to the exposed ends of the electric wires instead of the metallizing process.

The connector housing, such as connector housing 106 in FIG. 1, can be connected to an instrument, such as a computing device (e.g. a laptop, desktop, or tablet computer) or a physiology monitor, that converts the signals received by the sensors into pressure and velocity readings. The instrument can further calculate Coronary Flow Reserve (CFR) and Fractional Flow Reserve (FFR) and provide the readings and calculations to a user via a user interface.

In some embodiments, a user interacts with a visual interface to view images from the imaging system. Input from a user (e.g., parameters or a selection) are received by a processor in an electronic device. The selection can be rendered into a visible display. An exemplary system including an electronic device is illustrated in FIG. 12. As shown in FIG. 12, a sensor engine 859 communicates with host workstation 433 as well as optionally server 413 over network 409. The data acquisition element 855 (DAQ) of the sensor engine receives sensor data from one or more sensors. In some embodiments, an operator uses computer 449 or terminal 467 to control system 400 or to receive images. An image may be displayed using an I/O 454, 437, or 471, which may include a monitor. Any I/O may include a keyboard, mouse or touchscreen to communicate with any of processor 421, 459, 441, or 475, for example, to cause data to be stored in any tangible, nontransitory memory 463, 445, 479, or 429. Server 413 generally includes an interface module 425 to effectuate communication over network 409 or write data to data file 417.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data.

Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server 413), a middleware component (e.g., an application server), or a front-end component (e.g., a client computer 449 having a graphical user interface 454 or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a portion of file 417 that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A sensing guidewire, the guidewire comprising:
a core member sized to fit within a vessel;
a sensor coupled to the core member; and
a centering element comprising a retracted and deployed configuration, the centering element being coupled to the core member such that upon deployment of the centering element, the sensor on the core member is located within a center of a lumen.

2. The guidewire according to claim 1, wherein the centering element surrounds the guidewire such that it deploys a same distance on all sides of the guidewire.

3. The guidewire according to claim 1, wherein the centering element comprises a plurality movable struts.

4. The guidewire according to claim 1, wherein the sensor is positioned on a distal portion of the guidewire.

5. The guidewire according to claim 4, wherein the centering element is positioned on a distal portion of the guidewire.

6. The guidewire according to claim 1, wherein the sensor is a pressure sensor.

7. The guidewire according to claim 6, wherein the pressure sensor comprises a crystalline semi-conductor material.

8. The guidewire according to claim 1, wherein the sensor is a flow sensor.

9. The guidewire according to claim 8, wherein the flow sensor comprises ultrasound transducer

10. The guidewire according to claim 1, wherein the guidewire comprises a pressure sensor and a flow sensor.

11. A method for measuring a characteristic inside a vessel, the method comprising:
providing a sensing guidewire that comprises: a core member sized to fit within a vessel;
a sensor coupled to the core member; and a centering element comprising a retracted and deployed configuration, the centering element being coupled to the core member such that upon deployment of the centering element, the sensor on the core member is located within a center of a lumen;
inserting the guidewire into a vessel;
centering the sensor within the vessel via deployment of the centering element; and
using the sensor to measure a characteristic inside the vessel.

12. The method according to claim 11, wherein the centering element surrounds the guidewire such that it deploys a same distance on all sides of the guidewire.

13. The method according to claim 11, wherein the centering element comprises a plurality movable struts.

14. The method according to claim 11, wherein the sensor is positioned on a distal portion of the guidewire.

15. The method according to claim 14, wherein the centering element is positioned on a distal portion of the guidewire.

16. The method according to claim 11, wherein the sensor is a pressure sensor and the characteristic measured is intraluminal pressure.

17. The method according to claim 16, wherein the pressure sensor comprises a crystalline semi-conductor material.

18. The method according to claim 11, wherein the sensor is a flow sensor and the characteristic measured is intraluminal flow.

19. The method according to claim 18, wherein the flow sensor comprises ultrasound transducer

20. The method according to claim 11, wherein the guidewire comprises a pressure sensor and a flow sensor.
